(19) United States
(12) Patent Application Publication
(10) Pub. No.: US 2006/0292553 A1
(43) Pub. Date: Dec. 28, 2006

(54) Method for study of the genetic and functional variability of HIV and kit for using it

(75) Inventors: Sophie Lebel-Binay, Villejuif (FR); Elisabeth Dam, Paris (FR); Luc Boblet, Boutigny S/Opton (FR); Dominique Costantini, Paris (FR)

Correspondence Address: IP GROUP OF DLA PIPER US LLP, ONE LIBERTY PLACE, 1650 MARKET ST, SUITE 4900, PHILADELPHIA, PA, 19103, US

(73) Assignee: Bioalliance Pharma, a corporation of France, Paris (FR)

(21) Appl. No.: 11/073,972

(22) Filed: Mar. 7, 2005

(30) Foreign Application Priority Data
Apr. 16, 2004 (FR) FR 04/04039

(51) Int. Cl. C12Q 1/70; C12Q 1/68; C12P 19/34

(52) U.S. Cl. 435/5; 435/6; 435/91.2

(57) ABSTRACT

A method of analyzing a sample possibly containing an HIV virus, including a) extracting viral RNA in a biological sample that possibly contains an HIV virus; b) reverse transcription of the RNA obtained of (a) and amplification with a first pair of primers to obtain an amplified product of reverse transcription including all or part of at least two successive genes of a genome of an HIV virus; and one or both of c) and d): c) sequencing the amplified product of (b) to establish a genotype of HIV virus present in the sample and identify mutations that may be present in the amplified product; d1) amplifying the product of (b) with a second pair of primers complementary to the first pair of (b) and capable of generating an amplification product that can be inserted by homologous recombination into a retroviral vector that is defective in a region corresponding to the amplified product; d2) homologously recombining the product of (d1) with the defective vector; d3) functionally analyzing the viral proteins coded by all or part of the at least two successive genes of the product of (d1); and d4) measuring replicating capacity of recombinant viruses of (d2) in the presence or in the absence of at least one active substance.

### RELATED APPLICATION

This application claims priority of French Patent Application No. 04/04039, filed Apr. 16, 2004, herein incorporated by reference.

### FIELD OF THE INVENTION

This invention relates to the area of virus analysis for human immunodeficiency virus type 1 (HIV-1). In particular, the invention relates to a method and (its implementation means) for investigating the genetic and functional variability of HIV.

### BACKGROUND

Human immunodeficiency virus of the type 1 (HIV-1) is a coated retrovirus of which the genome codes, in particular, for three distinct enzymes: inverse transcriptase that transcribes viral RNA into double strand DNA; integrase, which permits the integration of the viral DNA into the genome of the target cell; and protease, which is necessary for maturation of the virions. The viral enzymes, inverse transcriptase (RT) and protease (PR) have become the main targets of the anti-retroviruses.

Currently, about 15 anti-retroviral molecules that inhibit inverse transcriptase and protease are used in clinical practice. The combined use of these inhibitors leads to great decreases in viral replication. However, these combinations of drugs are sometimes complicated by significant secondary effects, low compliance and development of viral strains that are resistant to anti-retroviruses.

One of the causes of failure of treatments for human immunodeficiency virus (HIV) is the emergence of mutant viruses that are resistant to antiviral treatments, which appear when suppression of viral replication is incomplete. Exhaustion of the pressure exercised by the drugs involves the appearance of mutations in the enzymes and viral proteins (RT and PR), which plays a significant role in replicating viral infectious ability (fitness). Resistant viruses may have a reduced infectious capability in comparison to “wild-type” viruses. Clinically, it appears important to have available, at the same time, information on the genetic and functional variability of the two main targets since these treatments are combined in clinical practice. Currently, no such tool is available. Starting with the same viral sample of the patient, current tests are not adequate to simultaneously explore known and unknown mutations and the infectious strength on at least two genetic targets of interest in the presence or absence of drugs.

Different current diagnostic tests make it possible to judge either the genetic variability (genotype) or the functional variability (phenotype and replicating capacity) of the viruses of patients infected with HIV to antiviruses (FIG. 1):
Tests of genotypical resistance
Tests of phenotypical resistance
Tests of viral replication
Combined tests

Viral RNA derived from the plasma is extracted, then the regions coding for inverse transcriptase and protease are analyzed in tests of genotypical resistance.

Currently, their method of analysis is based on the position of the amino acids, preceded and followed by a letter indicating the “wild type” amino acid and the mutant, respectively. For example, for a mutation of resistance to lamivudine M184V, valine replaces methionine in position 184 of the RT.

The majority of the current tests use techniques of automatic sequencing of the target genes. These tests detect the mutations present in the sequenced region, but are not able to interpret all of them. In fact, only known mutations are interpreted as a function of algorithms that are updated regularly by international committees of experts. These algorithms have become more and more complex with the treatment combinations and more than 15 drugs available on the market.

Other tests such as the “Line Probe Assay” (LiPA) or “Gene Chips” recommended by the Affymetrix Company are based on techniques of hybridization and use specific probes limited to identifying certain mutations.

Interpretation of the results of genotypical tests is complex because of the difficulty of estimating the cumulative effects of multiple mutations of which some may have additive effects, while others will restore their sensitivity.

The principle of the phenotypical tests for resistance is based on the measurement in vitro of the growth of a virus in the patient in the presence of drugs.

Viral RNA derived from the plasma is extracted, then the regions coding for inverse transcriptase and/or protease are amplified using PCR in phenotypical tests. Amplicons are recombined in vitro in a defective vector to form a viral particle. This viral particle is placed in a culture in the presence of increasing concentrations of drugs. The results are expressed in a “fold change” ratio of IC 50 (or IC90) with respect to a control virus which corresponds to the concentration of the drug that inhibits 50% (or 90%) of the viral replication in comparison to the reference wild type virus. The resistance level is defined as a function of thresholds of sensitivity (cut off).

Three main phenotypical tests for resistance are currently available: PhenoSense™ (Virologic, USA), Antivirogram™ (Virco, Belgium) and Phenoscript™ (VIRalliance, France). These tests give information on the susceptibility of drugs with respect to their target, but do not forecast the impact of sentry mutations on the evolution of the virus resistance.

It has been possible to combine the genotypical and phenotypical information to validate the technique. However, in that case, the methodology includes a step comprising construction of a recombination vector by ligation (Parkin et al. 2004, Antimicrob. Agents Chemother. 48:437) or requires multiple infection cycles for phenotyping (WO/0233638). These two technical approaches may introduce a bias in the representative nature of the virus of the patient.

On the other hand, the current phenotyping tests are not known to be compatible with the genotyping tests in use except for internal use.

Mutations with resistance induced by protease inhibitors and inverse transcriptase inhibitors are known for modifying the replicating capacity of the HIV virus.

The tests with determination of the replicating capacity in vitro are based on the use of a recombinant plasmid, transfected, then amplified in cellular culture. After normalization of the virus quantity, the viral supernatant is used to infect new cells. The replicating capacity is then evaluated over a given period of time, corresponding to a single cycle or several cycles of replication, according to the methodology used. The replicating capacity of a mutated variant is expressed in a general manner comparable to that of a wild type variant.

The qualification of a virus with strong infectious capacity is currently disconnected from its genetic and functional variability using the same sample from the patient.

WO/0233638 describes the possibility of carrying out phenotyping and genotyping using the same amplification product. However, the phenotyping technique used does not describe a single cycle of viral replication. In a first period of time, a viral production is necessary within the permissive cells making possible, in a second period of time, reinfection of indicative cells to measure IC50 (WO/9727480). These steps are not representative of the initial viral populations of the patient due to the multiple cycles of infection without the selective pressure of the drugs with the risk of evolution of the initial virus.

WO 2004/003513 proposes a method of genotyping, phenotyping and, in addition, replicating capacity centered on constructing by ligation a recombination vector containing a reporter gene and the sequence studied. This method is also less representative of the reality of the behavior of the virus in the course of the infection of the patient. Cloning by ligation is interesting for yielding the recombination, but may introduce a bias into the selection of the initial viral populations of the patient.

Tests described in WO/0238792 make it possible to measure the infectious capacity (replicating capacity) of the phenotype using the same recombination vector. In particular, the large fragment (>2800 pb) coding for a part of the gag gene and the regions of reading scope of the pol gene coding for protease and inverse transcriptase can be used to determine the replicating capacity of the virus. Still, this large fragment, in current practice, does not make it possible to obtain a rate of amplification success and a rate of replication sufficient to allow the measurement of the infectious capacity. The following steps thus cannot be carried out, which limits the use of this large fragment.

The known methods for studying the HIV virus are limited by the difficulty of simultaneously implementing a good representative nature of the behavior of the virus (homologous recombination) and an adequate level of amplification and replication including for very mutated viruses. It would therefore be advantageous to have a method to make it possible to study and better interpret the data of the virus and the data of the treatment of the patient using the same sample. In fact, there exists today an important need for a strategy that makes possible, using a single biological sample from a patient suffering from HIV, to obtain a measurement of genotypical resistance, phenotypical resistance and replicating capacity of the virus.

### SUMMARY OF THE INVENTION

This invention relates to a method of analyzing a sample possibly containing an HIV virus, including a) extracting viral RNA in a biological sample that possibly contains an HIV virus, b) reverse transcription of the RNA obtained in (a) and amplification with a first pair of primers to obtain an amplified product of reverse transcription including all or part of at least two successive genes of a genome of an HIV virus, and one or both of (c) and (d1-d4): (c) sequencing the amplified product of (b) to establish a genotype of HIV virus present in the sample and identify mutations that may be present in the amplified product, (d1) amplifying the product of (b) with a second pair of primers complementary to the first pair of (b) and capable of generating an amplification product that can be inserted by homologous recombination into a retroviral vector that is defective in a region corresponding to the amplified product, (d2) homologously recombining the product of (d1) with the defective vector, (d3) functionally analyzing the viral proteins coded by all or part of the at least two successive genes of the product of (d1), and (d4) measuring replicating capacity of recombinant viruses of (d2) in the presence or in the absence of at least one active substance.

### BRIEF DESCRIPTION OF THE DRAWINGS

Other advantages and characteristics of the invention will be seen in the examples that follow where reference will be made to the drawings attached, in which:

FIG. 1 is a flow chart, in table form of the main steps of the genotype, of the phenotype and of the replicating capacity analyses;

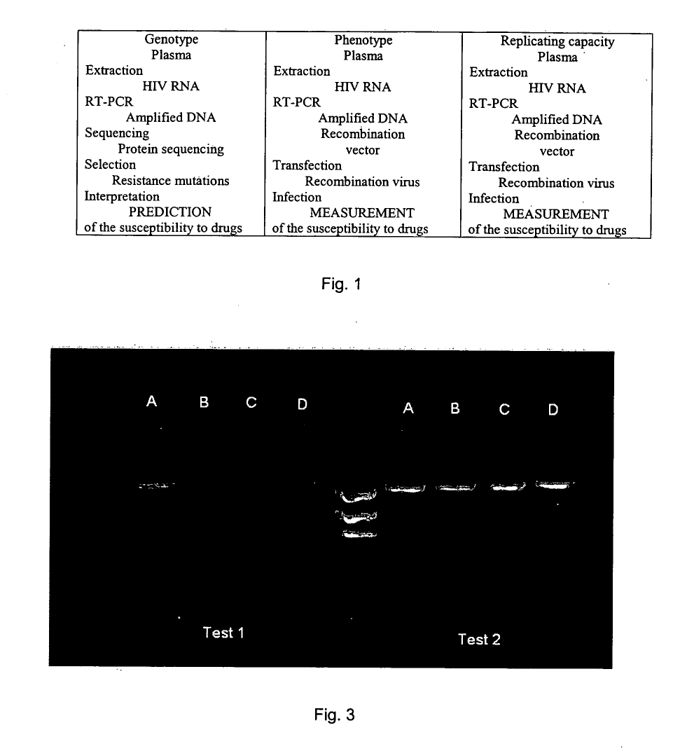

FIG. 2 is a flow chart showing the compatibility of the analyses of genotype, phenotype and replicating capacity according to aspects of the method;

FIG. 3 is a flow chart showing amplification of the viral populations: the RNA coming from four patients (A, B, C, D) have been retrotranscribed and amplified under the conditions described in WO 0238792 (test 1) or under conditions defined by aspects of this invention in Example 1 (test 2);

FIG. 4 is a graph showing the curve DO/P24 obtained in a test of replicating capacity for a patient virus (virus 1) and a reference virus;

FIG. 5 shows the position of pairs of primers according to aspects of the invention on the HIV genome and the organization of the gag and pol genes of the HIV-1:
Gag gene:
   Matrix: p17
   Core: p24
   Nucleocore (CA): p2, p7, p1, p6
Pol gene:
   Protease (PR): p10 cleavage and gag and pol maturation
   Reverse transcriptase (RT): p51: inverse transcription
   RNAse H: p15: RNAse H activity
   Integrase (Int): p3 1: proviral DNA integration; and

FIG. 6 shows regions amplified by different commercial tests for genotyping and phenotyping described in Example 3.

### DETAILED DESCRIPTION

This invention offers a new strategy that makes it possible, using a single biological sample from a patient infected with HIV, to obtain a measurement of genotypical and phenotypical resistance and the replicating capacity of the virus in such a way as to have a better understanding of the patient's situation and, thus, to be able to produce a better therapeutic orientation.

The invention thus provides a method of an analyzing sample likely to contain an HIV virus, comprising:
a) extracting viral RNA in a biological sample likely to contain an HIV virus;
b) reverse transcripting the RNA and amplifying the RNA with a first pair of primers to obtain an amplified product of inverse transcription comprising all or a portion of at least two successive genes of a genome of an HIV virus; and one or both of c) and d1-d4):
c) sequencing the amplified product to establish a genotype of the HIV virus present in the sample and identifying mutations present in the amplified product;
d1) amplifying the product of inverse transcription with a second pair of primers complementary to the first pair and capable of generating an amplification product that can be inserted by homologous recombination into a retroviral vector that is defective in a region corresponding to the amplified product;
d2) homologously recombining the amplified product with the defective vector;
d3) functionally analyzing the viral proteins coded by all or part of the at least two successive genes of the recombined product; and
d4) measuring the replicating capacity of the recombinant viruses in the presence or in the absence of at least one active substance.

The method according to the invention is remarkable in that it offers the possibility to measure the impact of anti-retroviral treatments, simultaneously judged on:

the genetic variation registering the known mutations, the combined mutations and the unknown mutations;

the functional variation recording the infectious capacity or replicating capacity of the virus in the presence or absence of anti-retrovirals; and

it makes it possible to simultaneously study, on the same biological sample from a patient, the impact of an antiviral agent on the genetic and functional variability on its initial target, e.g., the viral enzyme for which the anti-retroviral was designed, but also the tool will give information in parallel on the same data, genetic and functional variability, on one or more targets of interest.

The method according to the invention also makes it possible to be very representative of the behavior of the virus of the patient, it is also representative for evaluating the genetic and functional variability of one or several targets of the HIV-1 belonging to the subtypes B and non-B.

Each parameter, genotype, phenotype, replicating capacity, taken individually, adds to the information on the resistance and, according to the invention, the virus tested is the closest to its natural behavior. The invention makes it possible to measure three key parameters of resistance: genotype, phenotype and replication capacity using the same biological sample. It improves efficiency in isolating viral populations, makes possible the normalization of reconstituted viruses and a quantitative analysis of the results. The three aspects allow a better clinical interpretation and a reciprocal clarification of these items which makes it a combined tool for clinical use.

The method according to the invention more specifically concerns a method for analysis of samples that are apt to contain the HIV virus belonging to the sub-types B and non-B. Thus, the RNA is that of an HIV virus belonging to the sub-types B and non-B.

To carry out step (a), the method employs samples derived from a patient. It may be a blood or serum sample, but it may also come from a biological fluid or from a biopsy or from any other tissue preparation. The biological sample may also come from a viral culture. In a general manner, a biological sample corresponds to all types of samples containing one or more variations of HIV, in particular, HIV-1. The term HIV-1 virus is understood, as indicated above, as any viral strain belonging to the sub-types B and non-B.

To carry out step (b), the method employs a first pair of primers making it possible to obtain an amplified product of inverse transcription, also designated amplicon in the following, comprising all or part of at least two useful genes in the study of resistance to anti-retrovirals.

Thus, step (b) uses a pair of primers that makes it possible to prepare an amplicon characterized by:

the presence, at each of its ends, of conserved zones to allow amplification of the viral populations;

the potential presence of mutations of interest.

In one aspect of step (b), the use of a first pair of primers makes it possible to obtain an amplicon comprising all or part of the gag gene and of the pol gene coding for the protease and the inverse transcriptase involved in the replicating capacity of the virus and able to confer on the virus a resistance to treatment.

Thus, in step (b) it is a matter of obtaining an amplicon also having the following characteristics:

one part of the nucleic acid sequence coding for gag and including the cleavage sites,
all of the sequence coding for protease,
the sequence coding for inverse transcriptase going up to at least codon 340.
The amplicon defined above has a size less than about 2800 pb, preferably between about 2200 and about 2700 pb and most preferably between about 2300 and about 2600 pb.

Thus, advantageously, amplification in step (b) makes use of a pair of primers encompassing a nucleic sequence complementary at 5′ to the phylogenetically conserved region of the gag gene including the cleavage sites, containing all of the nucleic acid sequence coding for protease and complementary at 3′ to the phylogenetically conserved region of the gene coding for inverse transcriptase.

In one aspect, the pair of primers used in step (b) encompasses a nucleic acid sequence:

complementary at 5′ to the phylogenetically conserved region of the gag gene included between codon 102 of protein p17 (position 1093 on the genome) and codon 76 of protein p24 (position 1415 on the genome), and

complementary at 3′ to the phylogenetically conserved region of the gene coding for inverse transcriptase, included between codon 325 (position 3520) and codon 421 (position 3811 on the genome).

In another aspect, the pair of primers used in step (b) encompasses a nucleic acid sequence:

complementary at 5′ to the phylogenetically conserved region of the gag gene included between codon 126 (position 1165 on the genome) of protein p17 and codon 21 of protein p24 (position 1250 on the genome), and

complementary at 3′ to the phylogenetically conserved region of the gene coding for inverse transcriptase included between codon 335 (position 3550 on the genome) and codon 395 (position 3751 on the genome).

The amplification in step (b) is carried out with a pair of primers having a size between about 10 and about 50 nucleotides, preferably between about 20 and about 30 nucleotides.

Advantageously, the amplification in step (b) is carried out with a pair of primers chosen from the group comprising:

as a sense primer, those represented by one of the sequences SEQ ID NO. 1, SEQ ID NO. 3 and SEQ ID NO. 5 (Table 1),

as an anti-sense primer, those represented by one of the sequences SEQ ID NO. 2, SEQ ID NO. 4 and SEQ ID NO. 6 (Table 1),

fragments or analogues of these sequences.

The term “analogue” is understood to mean either sequences having one or several mutations without these altering the hybridization capacities under the strict conditions generally encountered at the time of PCR, or sequences that are located from 1 to 10, 1 to 5 or 1 to 3 nucleotides upstream or downstream of the sequences of primers.

Most especially, the invention concerns the use at step (b) of a pair of primers chosen from the group comprising:
the pair of primers R1 of sequences SEQ ID NO. 1 and SEQ ID NO. 2 in Table 1,
the pair of primers R2 of sequences SEQ ID NO. 3 and SEQ ID NO. 4 in Table 1,
the pair of primers R3 of sequences SEQ ID NO. 5 and SEQ ID NO. 6 in Table 1.

Step (c) of sequencing of the method makes it possible to identify known, unknown and combined mutations using the data available in the literature.

In step (d1), amplification of the amplified product of inverse transcription obtained in step (b) uses a pair of primers which makes it possible to prepare an amplicon characterized by:

the presence at each of its ends of conserved zones to allow recombination with the retroviral vector,

the potential presence of mutations of interest.

In step (d1), amplification of the amplified product of inverse transcription obtained in step (b) comprising all or part of the gag gene and of the pol gene coding for the protease and the inverse transcriptase is advantageously carried out with a second pair of primers, complementary to the first pair used in step (b), making it possible to obtain an amplicon that additionally comprises:

part of the sequence of nucleic acids coding for gag and including the cleavage sites,

the sequences coding for the protease,

the sequence coding for the inverse transcriptase going at least up to codon 340.

The amplicon as defined above has a size less than about 2800 pb, preferably between about 2200 and about 2700 pb and most preferably between about 2300 and about 2600 pb.

Thus, advantageously, the amplification in step (d1) uses a pair of primers including a sequence of nucleic acids, complementary at 5′ to the phylogenetically conserved region of the gag gene including the cleavage sites, containing all of the nucleic acid sequence coding for the protease, complementary at 3′ to the phylogenetically conserved region of the gene coding for the inverse transcriptase.

Most especially, the pair of primers used in step (d1) includes a sequence of nucleic acids:

complementary at 5′ to the phylogenetically conserved region of the gag gene included between codon 102 of the protein p17 (position 1093 on the genome) and codon 76 of the protein p24 (position 1415 on the genome), and

complementary at 3′ to the phylogenetically conserved region of the gene coding for inverse transcriptase, included between the codon 325 (position 3520) and the codon 421 (position 3811 on the genome).

In a most preferred manner, the pair of primers used in step (d1) includes a sequence of nucleic acids:

complementary at 5′ to the phylogenetically conserved region of the gag gene included between codon 126 (position 1165 on the genome) and protein p17 and codon 21 of protein p24 (position 1250 on the genome), and

complementary at 3′ to the phylogenetically conserved region of the gene coding for inverse transcriptase included between codon 335 (position 3550 on the genome) and codon 395 (position 3751 on the genome).

The amplification in step (d1) is carried out with a pair of primers having a size between about 10 and about 50 nucleotides, preferably between about 20 and about 30 nucleotides.

Advantageously, the amplification in step (d1) is carried out with a pair of primers chosen from among the group comprising:

as a sense primer, those represented by one of the sequences SEQ ID NO. 7 and SEQ ID NO. 9 (Table 4),

as an anti-sense primer, those represented by one of the sequences SEQ ID NO. 8 and SEQ ID NO. 10 (Table 4),

fragments or analogues of the sequences.

The term “analogue” in this connection is understood to mean either sequences having one or several mutations without this altering the hybridization capacities under the strict conditions generally encountered at the time of PCR, or sequences that are located from 1 to 10, 1 to 5 or 1 to 3 nucleotides upstream or downstream of the said primer sequences.

In one aspect, the invention concerns the use at step (d1) of a pair of primers chosen from the group comprising:
the pair of primers N1 with sequences SEQ ID NO. 7 and SEQ ID NO. 8 in Table 4,
the pair of primers N2 with sequences SEQ ID NO. 9 and SEQ ID NO. 10 in Table 4.

The functional analysis of step (d3) of one aspect of the method comprising infecting the HIV target cells with recombinant viruses produced in step (d2) in the presence or in the absence of one or more active agents. By way of example, it is possible to make reference to the infection of target HIV cells, in the presence or in the absence of several drugs, containing an indicator gene, independent of a retroviral vector, of which the expression is connected to the viral infection.

Measurement of the replicating capacity in step (d4) in one aspect of the method comprising measuring the expression of an indicator gene in response to the infection by the recombinant virus produced in step (d2) in comparison to a reference virus. By way of example, it is possible to make reference to the procedures integrating the optical density values derived from an enzymatic reaction connected with a relativizing gene independent of the vector and present in the infected cells.

According to one aspect, the method comprises, in the case where steps (c) and (d3) and (d4) have been carried out:
e) processing data relating to:
the possible presence of mutations determined at step (c), and
functional analysis of the viral proteins of step (d3), and
the replicating capacity of step (d4), to obtain the characteristics of the virus and/or of the treatment.

By way of example of data processing of this type, it is possible to make reference to interpretation algorithms for the mutations identified in step (c), the values of the concentrations of drugs inhibiting 50% (IC50) or 90% (IC90) of the viral replication obtained in step (d3), the comparison of the replicating capacity of the recombinant virus in step (d4) with a reference virus in the absence of drugs. These make possible a reciprocal interpretation of the characteristics of the virus and the treatment in such a way as to provide a new tool for individual and collective epidemiological tracking.

The invention also concerns the primers and combinations of them for amplification of the sequences of nucleic acids of HIV as defined above.

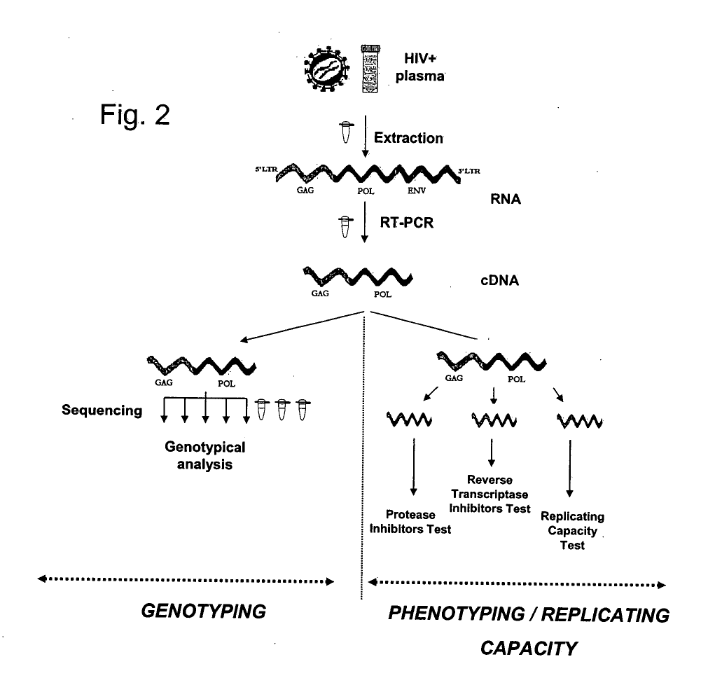

The method makes it possible to provide a new test that is capable, using the same biological sample from an HIV patient, to obtain a measurement of the genotypical, phenotypical resistance and the replicating capacity of the virus, i.e. the genetic variation recording the known mutations, the combined mutations and the unknown mutations and the functional variation recording the infective strength or replicating capacity of the virus in the presence or in the absence of anti-retroviruses (FIG. 2).

The invention thus also concerns a kit to perform the methods described above comprising one or several primers defined above. Such a kit also comprises a method of analyzing the three types of data obtained due to the method: genotype, phenotype and replicating capacity and the like.

### EXAMPLE 1

### Genetic and Functional Analysis of the Protease and the Inverse Transcriptase

The method in one aspect comprises generation of a specific nucleic acid simultaneously compatible with the genotyping and phenotyping techniques.

The procedure to generate the first specific amplicon is as follows:
1) Viral RNA contained in a biological sample is extracted. The biological sample may be derived from a sample from the patient. It may be a blood or serum sample, but may also come from a biological fluid or a biopsy or any other tissue preparation. The biological sample may also come from a viral culture. In a general manner, a biological sample corresponds to all types of samples containing one or several HIV-1 variants. The term “HIV-1 virus” in this Example is used to mean any viral strain belonging to the sub-types B and non-B.
2) The RNA obtained in (1) is retrotranscribed and amplified with one of the pairs of specific primers in Table 1 below to obtain an amplicon characterized by:

the presence, at 5′ and at 3′, of conserved zones to allow amplification of the viral populations;

the presence of any of the mutations of interest that have already been described;

the presence of part of the sequence of nucleic acids coding for gag and including the cleavage sites;

all of the sequence coding for the protease;

the sequence coding for the inverse transcriptase going to at least codon 340.

TABLE 1

| RT- | Size | | |
|---|---|---|---|
| PCR | Pb | Name | Sequence |
| Pair | 2379 | FIT ex+ | CCTCCAggggCAAATggTACATCA |
| R1 | | | (SEQ ID NO. 1) |
| | | FIT ex− | CTTgATAAATTTgATATgTCCATTggCCTT |
| | | | (SEQ ID NO. 2) |
| Pair | 2358 | GP A+ | TCACCTAgAACTTTAAATgC |
| R2 | | | (SEQ ID NO. 3) |
| | | RT A− | TTAAATggCTCTTgATAAATTTgA |
| | | | (SEQ ID NO. 4) |
| Pair | 2586 | GP B+ | AgCCAggTCAgCCAAAATTA |
| R3 | | | (SEQ ID NO. 5) |
| | | RT B− | CATgCTTCCCATgTTTCCTT |
| | | | (SEQ ID NO. 6) |

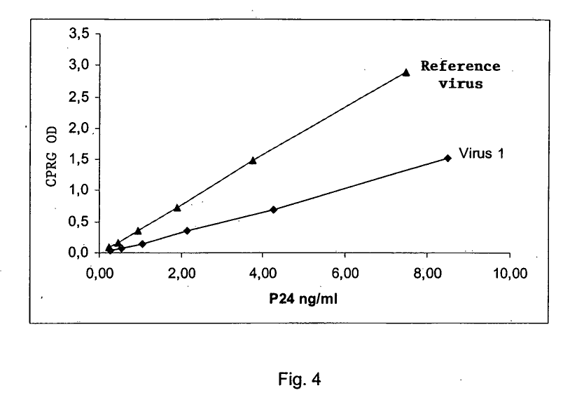

FIG. 3 shows that, in four patients, the method provides a better amplification of the viral populations than the conditions in the prior patent. On the other hand, on a series of 26 patients, the method allows amplification of all of the sample while the method described in WO 02/38792 allows only effective amplification of 16 samples of the 26 tested (four are negative and six are too weakly amplified).

Tables 2 and 3 below show two examples of samples of plasma in patients 1 and 2, respectively, for which the amplicons generated by the method above have made it possible to implement the genotyping according to the Trugene and Viroseq techniques and phenotyping according to the Phenoscript technique.

TABLE 2

| | | Patient 1 | | |
|---|---|---|---|---|
| | | | | Phenoscript |
| | | | | Contribution |
| List of | | Viroseq | Trugene | to the |
| mutations | | Evidence of | Interpretation of | therapeutic |
| identified | Drugs | resistance | resistances | response |
| NRTI | NRTI | | | |
| D67N | AZT | High | Resistance | Improbable |
| K70R | 3TC | High | Resistance | Improbable |
| M184V | ddC | High | nd | Nd |
| T215Y | ddI | Possible | Resistance | Probable |
| K219E | d4T | High | Resistance | Probable |
| | ABC | Possible | No resistance | Possible |
| | TDF | nd | No resistance | Nd |
| NNRTI | NNRTI | | | |
| K103N | DLV | High | Resistance | Nd |
| V108I | EFV | High | Resistance | Improbable |
| P225H | NVP | High | Resistance | Improbable |
| IP | IP | | | |
| L63P | APV | Zero | No resistance | Probable |
| | IDV | Zero | No resistance | Probable |
| | SQV | Zero | No resistance | Probable |
| | LPV | Zero | No resistance | Probable |
| | RTV | Zero | No resistance | Nd |
| | NFV | Zero | No resistance | Probable |

TABLE 3

| | | Patient 2 | | |
|---|---|---|---|---|
| | | | | Phenoscript |
| | | | | Contribution |
| List of | | Viroseq | Trugene | to the |
| mutations | | Evidence of | Interpretation | therapeutic |
| identified | Drugs | resistance | of resistances | response |
| NRTI | NRTI | | | |
| M41L | AZT | High | Resistance | Improbable |
| M184V | 3TC | High | Resistance | Improbable |
| T215Y | ddC | High | nd | Nd |
| L210W | ddI | Possible | Resistance | Possible |
| | d4T | Possible | Resistance | Probable |
| | ABC | Possible | Resistance | Possible |
| | | | possible | |
| | TDF | nd | Resistance | Nd |
| NNRTI | NNRTI | | | |
| | DLV | Zero | No resistance | Nd |
| | EFV | Zero | No resistance | Probable |
| | NVP | Zero | No resistance | Probable |
| IP | IP | | | |
| M36L | APV | Zero | No resistance | Probable |
| L63P | IDV | Zero | No resistance | Probable |
| | SQV | Zero | No resistance | Probable |
| | LPV | Zero | No resistance | Probable |
| | RTV | Zero | No resistance | Nd |
| | NFV | Possible | No resistance | Probable |

### EXAMPLE 2

### Analysis of the Replicating Capacity of the HIV Virus from Patients having Mutations in the Protease and the Inverse Transcriptase

In this example, the method comprises the following steps:
1) The viral RNA that is contained in the biological sample is extracted.
2) The RNA obtained in (1) is retrotranscribed and amplified with a pair of specific primers making it possible to amplify in a regular manner a specific amplicon comprising at least two genes of interest in the study of the resistance to anti-retroviruses, as described in Example 1.
3) A new amplicon using the amplicon obtained in step (2) above is prepared by using a new pair of primers described in Table 4 below. This new amplicon is characterized by:

the presence, at 5′ and at 3′, of conserved zones to allow recombination with the retroviral vector;

the presence of all the mutations of interest that have already been described;

the presence of part of the sequence of nucleic acids coding for gag and including the cleavage sites;

all of the sequence coding for protease;

the sequence coding for the inverse transcriptase going up to at least codon 340.

TABLE 4

| Nest | | Name of | |
|---|---|---|---|
| PCR | Size | primer | Sequence |
| Pair | 2360 | FIT in+ | TggTACATCAggCCATATCACCTAgAACTT |
| N1 | pb | | (SEQ ID NO. 7) |
| | | FIT in− | TAAATTTgATATgTCCATTggCCTTgCC |
| | | | (SEQ ID NO. 8) |
| Pair | 2338 | GP E+ | AgAACTTTAAATgCATgggT |
| N2 | pb | | (SEQ ID NO. 9) |
| | | RT E− | TAAATTTgATATgTCCATTggCCTT |
| | | | (SEQ ID NO. 10) |

The primers described in Table 4 make possible better recombination with the patients having numerous mutations with a new retroviral vector deleted from one part of the gag gene, of the region of the reading scope of the pol coding for the protease and a part of the inverse transcriptase of the HIV-1 than the recombination described under the conditions of WO 02/38792.

In Tables 5 and 6 below, the mutations identified in the gene of the protease and of the inverse transcriptase for a series of six patients are listed. Table 7 gives the average values of replicating capacity obtained for these six patients in two independent tests using the method. Under the conditions described in WO 02/38792, on this series of patients having numerous mutations, the recombination with the retroviral vector was not effective enough to produce a satisfactory level of recombinant viruses and make possible an analysis of the replicating capacity.

TABLE 5
List of main mutations identified for the protease
inhibitors (IP) on the six patients studied.

| IP | CR01 | CR02 | CR03 | CR04 | CR05 | CR06 |
|---|---|---|---|---|---|---|
| L10 | | I | I | I | I/F | I |
| K20 | I | | | | | |
| L24 | | | | | I | |
| M36 | | | | | I | |
| M46 | I | | L | L | I | L |
| G48 | | | V | | | |
| I54 | | | | V | V | |
| L63 | P | P | P | P | L/P | P |
| A71 | I | | V | T | | |
| G73 | | T | | | | |
| V77 | | I | I | I | | |
| V82 | | | A | A | A | A |
| I84 | V | V | | | | |
| L90 | M | | M | M | | M |

TABLE 6
List of the main mutations identified for the inverse transcriptase
inhibitors (RTI) in the six patients studied.

| RTI | CR01 | CR02 | CR03 | CR04 | CR05 | CR06 |
|---|---|---|---|---|---|---|
| M41 | L | | L | L | L | |
| E44 | D | | A | | D | |
| A62 | | V | | | | |
| D67 | N | | N | | N | N |
| T69 | | S + V + A | | | D | D |
| K70 | | | | | | R |
| L74 | | V | | | | |
| K103 | N | | | | N | |
| V118 | V/I | | V/I | | I | |
| Y181 | | C | I | | | |
| M184 | | | V | V | V | V |
| G190 | S | A | | | | |
| L210 | W | | W | | W | |
| T215 | Y | Y | Y/C | Y | Y | |
| K219 | N | K/Q | E | | | Q |

TABLE 7

| | Average of the % | | |
|---|---|---|---|
| Patients | of the reference | Standard deviation | CV % |
| CR 01 | 42.14 | 16.14 | 38.3 |
| CR 02 | 5.78 | 0.09 | 1.5 |
| CR 03 | 11.88 | 5.65 | 47.6 |
| CR 04 | 8.76 | 0.31 | 3.6 |
| CR 05 | 14.09 | 3.73 | 26.5 |
| CR 06 | 20.70 | 2.90 | 14.0 |

The primers described in Table 4 also make possible, on another series of four patients, production of a quantity of recombinant viruses that is more significant than the recombination described under the conditions of WO 02/38792 as well as the normalization of the infection of the indicative cells by using, for example, the dosage of antigen p24. Table 8 below describes that the quantity of p24 produced by the recombinant viruses of four patients according to this method (GRF vector) is greater than that of WO 02/38792 (GPR vector).

TABLE 8

| Patients | Vector | Quantity of p24 (ng/ml) |
|---|---|---|
| BA 01 | GPR | 115 |
| BA 01 | GRF | 261 |
| BA 02 | GPR | 108 |
| BA 02 | GRF | 132 |
| BA 03 | GPR | 98 |
| BA 03 | GRF | 280 |
| BA 04 | GPR | 195 |
| BA 04 | GRF | 430 |

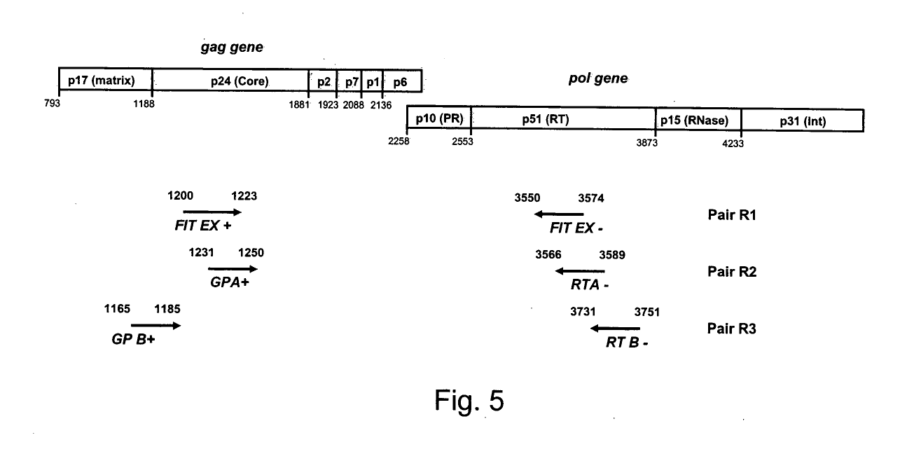

The primers in Table 4 again make it possible to measure the replicating capacity over a range of recombinant viruses in comparison to a reference virus according to the quantitative methods integrating the optical density values resulting from an enzymatic reaction connected with a revealing gene independent of the vector and present in the infected cells (FIG. 4).

The primers in Table 4 make it possible to obtain good reproducibility of the measurement of the replicating capacity on two control samples, of which one has mutations known to decrease the replicating capacity. Table 9 below shows the reproducibility of the value of replicating capacity on two control samples having mutations known in the protease. Three independent tests.

TABLE 9

| | | Average of | | |
|---|---|---|---|---|
| | Mutations in the | the % of | Standard | |
| Samples | protease | the reference | deviation | CV % |
| GPCA | L10I, G48V, V82A | 17.3 | 2.1 | 12.3 |
| GPCB | I54V, A71V, V82A | 46.8 | 3.1 | 6.6 |

### EXAMPLE 3

### Compatibility of the Amplicon of Example 1 and 2 with Different Commercial Tests

1) Compatibility with Main Commercial Tests for Genotyping

Four main commercial tests are described in an article by W. Cavert and H. H. Balfour (Detection of antiretroviral resistance in HIV-1 Clin Lab Med 2003 23:915).

1.1) Trugene kit (Bayer Visible Genetics Inc.) (WO 02/070731; Grant et al. Accuracy of the Trugene HIV-1 Genotyping kit J Clin Microbiol 2003 41:1586)

The Trugene HIV-1 genotyping kit is used to determine the genotype of the virus of sub-types B and non-B. The RNA is extracted using plasma from patients, according to known techniques, the viral RNA is retrotranscribed and amplified using PCR with primers specific for the pol gene making possible amplification of a sequence of nucleic acids of 1300 pb comprising, for the protease, codons 1 to 99 and comprising, for the inverse transcriptase, codons 1 to 247. The product of RT-PCR thus obtained is used in each of the 16 sequencing reactions by using the principle of CLIP™ reaction, sequencing technique using labeled primers (dye primers). Four pairs of different primers are used for this sequencing method, two pairs for the sequencing of the protease and two other pairs for the sequencing of the inverse transcriptase. Each sequence reaction is initiated by a specific dye primer (CLIP™), then interrupted by the corresponding labeled nucleotide. All of the fragments synthesized are then separated on electrophoresis gel and analyzed by an automatic sequencer specifically indicating the fragments ending with each of the four labeled nucleotides. The sequences, once reconstituted, are compared to the sequence of a reference virus using an alignment software.

1.2) ViroSeq™ (Applied Biosystems) (* M et al. J Clin Microbiol. 2001. 39:4323)

The RNA is extracted using plasmas from patients according to a known extraction technique based on the affinity of RNA with respect to silica columns, the viral RNA is retrotranscribed and amplified using PCR with primers specific to the pol gene making possible amplification of a sequence of nucleic acids of 1800 pb comprising, for the protease, codons 1 to 99 and comprising, for the inverse transcriptase, codons 1 to 335. The DNA thus obtained is then sequenced with seven different primers according to the Big Dye™ Terminator technology, a sequencing technique using dye terminators. The sequence reaction is initiated by each specific non-labeled primer, then interrupted by each of the labeled nucleotides. All of the fragments synthesized are then separated on electrophoresis gel. The color of the fluorochrome will then be registered using an automatic sequencer (ABI Prism 377 DNA sequencer), specifically indicating the fragments ending with each of the four labeled nucleotides. The sequences, once reconstituted, are compared to the sequence of a reference virus using an alignment software.

1.3) GeneSeq™ (ViroLogic) (Parkin NT et al., Antimicrob. Agents Chemother. 2004. 48:437)

This technology uses resistance vectors constructed for the PhenoSense phenotypical test. The sequence of nucleic acids of the vector comprising, for the protease, the codons 1 to 99 and comprising, for the inverse transcriptase, codons 1 to 305 is analyzed by sequencing using different combinations of fluorescent probes.

The nucleic acids are then deposited on an electrophoresis gel and analyzed using an automatic sequencer. The sequences obtained are compared to those obtained for the reference viruses.

1.4) GenoSure™ (Virco) (WO 01/81624)

The RNA is extracted from patient plasmas according to known extraction techniques, the viral RNA is retrotranscribed and amplified using PCR with primers specific for the pol gene making possible amplification of a sequence of nucleic acids of 1800 pb comprising, for the protease, codons 1 to 99 and comprising, for the inverse transcriptase, codons 1 to 415. The DNA thus obtained is then sequenced according to Big Dye™ Terminator technology, as described above.

2) Compatibility with Main Commercial Phenotyping Tests

A recent summary by M. Youle, “Clinical Issues in HIV,” published in December 2003 at the site www.hivandhepatitis.com describes the main resistance tests.

Three tests are currently available: Phenoscript™ (Viralliance); Antivirogram™ (Virco); PhenoSense™ (ViroLogic).

2.1) The test developed by Virco, Antivirogram™, is not compatible with this method since the fragment of nucleic acids of 2200 pb amplified using the RNA of the patient comprises the protease of codon 10 to codon 99 and all of the inverse transcriptase. (Hertogs et al. 1998. Antimicrob. Agents Chemother)

2.2) PhenoSense (ViroLogic) (Parkin NT et al., Antimicrob. Agents Chemother. 2004. 48:437; Petropoulos et al., Antimicrob. Agents Chemother. 2000. 44:920)

The PhenoSense test is carried out using viral RNA extracted from plasma of an HIV patient. The regions of the pol gene coding for the protease and the inverse transcriptase are amplified using RT-PCR to obtain a sequence of nucleic acids of 1500 pb comprising the cleavage sites of the protein gag (p7-p1-p6), the entire region coding for the protease and the region of the inverse transcriptase going from codon 1 to codon 313. This sequence is then inserted by ligation into an HIV retroviral vector containing a reporter gene (luciferase) and deleted in the encapsulation protein of the HIV. The retroviral vector is then co-transfected into cells 293T with a vector coding for the encapsulation protein MLV (Murine Leukemia Virus). The viruses produced are used to infect new cells in the presence or in the absence of anti-retroviruses. The luciferase activity in the infected cells in the presence of drugs is compared to the luciferase activity in the absence of drugs which makes possible calculation of concentrations inhibiting 50% of the viral production (IC50).

Table 10 below summarizes the characteristics of the sequences of nucleic acids amplified in the main tests described above.

TABLE 10

| | Size of | | | |
|---|---|---|---|---|
| | amplified | | | Inverse |
| Test | fragment | gag | Protease | transcriptase |
| Trugene ™ | 1300 pb | nd | Codons 1-99 | Codons 1-247 |
| ViroSeq ™ | 1800 pb | nd | Codons 1-99 | Codons 1-335 |
| GeneSeq ™ | 1400 pb | nd | Codons 1-99 | Codons 1-305 |
| GenoSure ™ | 1800 pb | nd | Codons 1-99 | Codons 1-415 |
| PhenoSense ™ | 1500 pb | p7-p1-p6 | Codons 1-99 | Codons 1-313 |

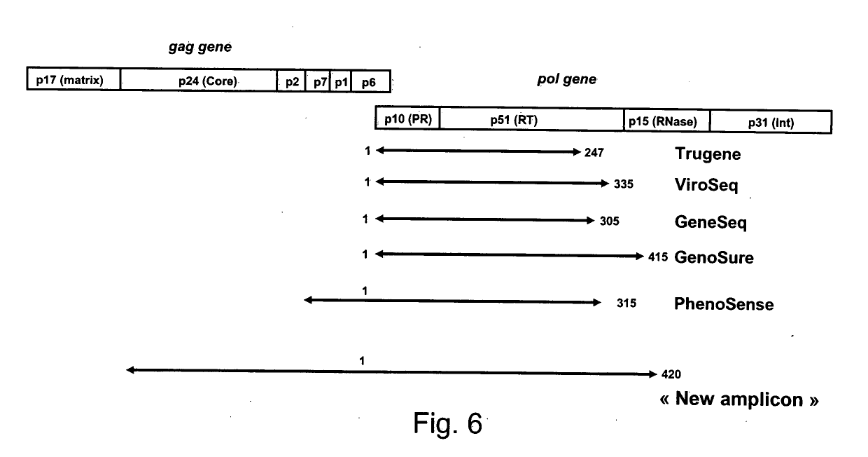

FIG. 6 shows the regions amplified by the different commercial tests for genotyping and phenotyping described in Example 3. The amplicon according to aspects of this invention, designated “new amplicon” in FIG. 6, is compatible with all of these tests.

### EXAMPLE 4

### Determination of a Score to use as a Tool in making the Treatment Decision

Using a biological sample from an HIV patient, this method makes it possible to take into account, in a scoring system, measurements of genetic and functional variability of an HIV virus belonging to the sub-types B and non-B.

The measurement of genetic variability is carried out using a specific sequence of nucleic acids amplified according to aspects of the method, then analyzed according to known sequencing techniques (Trugene, ViroSeq). Mutations in the genes of the protease and of the inverse transcriptase identified by these tests are interpreted according to algorithms that are updated regularly. A first score of 0 to 2 is attributed to each of the three levels of resistance determined by the interpretation. Table 11 below summarizes the interpretations given by the Trugene and ViroSeq tests as a function of the mutations identified and of the anti-retroviruses (ARV) used and assigns a genotypical score corresponding to the magnitude of the resistance.

TABLE 11

| | Trugene ™ | |
|---|---|---|
| ViroSeq ™ Evidence | Interpretation of | Genotypical |
| of Resistance | the resistance | score |
| High | Resistance | 0 |
| Possible | Resistance | 1 |
| | possible | |
| Zero | No resistance | 2 |

The measurement of the functional variability of an HIV virus comprises analysis of the replicating capacity of the virus in the presence (phenotype) or the absence of anti-retroviruses (fitness).

The principle of the tests of phenotypical resistance is based on the measurement in vitro of the growth of a virus in the patient in the presence of drug(s)/active agent(s), compared to a reference virus (resistance index). The level of resistance is defined as a function of thresholds of sensitivity (cut off). A second score of 0 to 2 is attributed to each of the three levels of resistance determined by the interpretation. Table 12 summarizes the interpretations given by the main phenotypical tests, PhenoSense and Phenoscript, as a function of their thresholds of sensitivity and assigns a phenotypical score corresponding to the magnitude of resistance.

Table 12 below summarizes assignment of a phenotypical score as a function of the interpretation of the phenotypical thresholds.

TABLE 12

| | Phenoscript ™ | |
|---|---|---|
| PhenoSense ™ Comparison | Contribution to the | Phenotypical |
| of sensitivity | therapeutic response | score |
| Less sensitive | improbable | 0 |
| Sensitive | possible | 1 |
| More sensitive | probable | 2 |

The replicating capacity of a virus, or fitness, in the absence of an anti-retrovirus is measured in comparison to a reference virus. It provides information on the capacity of the virus to replicate itself and is expressed in percent of reference virus. By way of example, there can be a virus having a fitness of 100%, which is considered as having a strong replicating activity, and a virus having a fitness of 10%, which is considered as having a low replicating activity.

The combination of the two scores, genotype, phenotype, and the percentage of the replicating capacity using the same biological sample must allow a better interpretation of the clinical data and supply a tool that is an aid to treatment decisions. In practice, addition of the genotypical and phenotypical scores for a given anti-retrovirus, combined with the measurement of the replicating capacity, can make it possible to orient the treatment choice. The genotypical plus phenotypical score is between 0 and 4 for each ARV and is accompanied by a percent of replicating capacity.

Thus, for an ARV in progress at the moment of sampling, if the genotypical plus phenotypical score is less than 1 and the replicating capacity is elevated (100% or more), it is necessary to promote the arrest of the molecule since the virus is resistant and maintains a strong capacity to replicate in the presence of this ARV.

For an ARV in progress at the moment of sampling, if the genotypical plus phenotypical score is less than 1 and the replicating capacity is low (10%), the pursuit of the molecule may be preferred to the discontinuation of treatment by the clinician in the absence of alternative treatment.

1) A method of analyzing a sample possibly containing an HIV virus, comprising:
a) extracting viral RNA in a biological sample that possibly contains an HIV virus;
b) reverse transcription of the RNA obtained in (a) and amplification with a first pair of primers to obtain an amplified product of reverse transcription comprising all or part of at least two successive genes of a genome of an HIV virus; and
one or both of c) and d1- d4):
c) sequencing the amplified product of (b) to establish a genotype of HIV virus present in the sample and identify mutations that may be present in the amplified product;
d1) amplifying the product of (b) with a second pair of primers complementary to the first pair of (b) and capable of generating an amplification product that can be inserted by homologous recombination into a retroviral vector that is defective in a region corresponding to the amplified product;
d2) homologously recombining the product of (d1) with the defective vector;
d3) functionally analyzing the viral proteins coded by all or part of the at least two successive genes of the product of (d1);
d4) measuring replicating capacity of recombinant viruses of (d2) in the presence or in the absence of at least one active substance.

2) The method according to claim 1, wherein the amplified product of reverse transcription comprises all or part of at least two genes useful in the study or resistance to anti-retroviruses.

3) The method according to claim 1, wherein the amplified product of reverse transcription has:
at each end thereof, conserved zones to allow amplification of the viral populations; and
potential presence of mutations of interest.

4) The method according to claim 1, wherein the amplified product comprises all or part of a gag gene and a pol gene coding a protease and reverse transcriptase involved in replicating capacity of the virus and able to confer on the virus a resistance to treatment.

5) The method according to claim 4, wherein the amplified product comprises:
one part of a nucleic acid sequence coding for gag and including cleavage sites,
all of the sequence coding for protease, and
a sequence coding for inverse transcriptase up to at least codon 340.

6) The method according to claim 1, wherein the amplified product has a size less than about 2800 pb, preferably between 2200 and 2700 pb and most preferably between 2300 and 2600 pb.

7) The method according to claim 1, wherein the amplified product has a size less than about 2800 pb.

8) The method according to claim 1, wherein the amplified product has a size between about 2300 and about 2600 pb.

9) The method according to claim 4, wherein the first pair of primers encompasses a nucleic sequence complementary at 5′ to a phylogenetically conserved region of the gag gene including cleavage sites, containing all of a nucleic acid sequence coding for protease and complementary at 3′ to a phylogenetically conserved region of a gene coding for inverse transcriptase.

10) The method according to claim 4, wherein the first pair of primers encompasses a nucleic acid sequence:
complementary at 5′ to a phylogenetically conserved region of the gag gene included between codon 102 of protein p17 (position 1093 on the genome) and codon 76 of protein p24 (position 1415 on the genome), and
complementary at 3′ to phylogenetically conserved region of the gene coding for inverse transcriptase, included between codon 325 (position 3520) and codon 421 (position 3811 on the genome).

11) The method according to claim 4, wherein the first pair of primers encompasses a nucleic acid sequence:
complementary at 5′ to a phylogenetically conserved region of the gag gene included between codon 126 (position 1165 on the genome) of protein p17 and codon 21 of protein p24 (position 1250 on the genome), and
complementary at 3′ to a phylogenetically conserved region of the gene coding for inverse transcriptase included between codon 335 (position 3550 on the genome) and codon 395 (position 3751 on the genome).

12) The method according to claim 4, wherein amplification is carried out with a pair of primers selected from the group consisting of:
SEQ ID NO. 1, SEQ ID NO. 3 and SEQ ID NO. 5 as a sense primer,
SEQ ID NO. 2, SEQ ID NO. 4 and SEQ ID NO. 6 as an anti-sense primer, and
fragments or analogues thereof.

13) The method according to claim 4, wherein the amplification is carried out with a pair of primers selected from the group consisting of:
a pair of primers R1 of sequences SEQ ID NO. 1 and SEQ ID NO. 2,
a pair of primers R2 of sequences SEQ ID NO. 3 and SEQ ID NO. 4, and
a pair of primers R3 of sequences SEQ ID NO. 5 and SEQ ID NO. 6.

14) The method according to claim 1, wherein the sequencing identifies known, unknown and combined mutations using data available in known literature.

15) The method according to claim 1, wherein amplification of (b) uses a pair of primers comprising:
conserved zones at ends of the primers that allow recombination with the retroviral vector, and
potential presence of mutations of interest.

16) The method according to claim 15, wherein amplification of the amplified product of inverse transcription of (b) comprises:
part of a sequence of nucleic acids coding for gag and including cleavage sites,
all of the sequence coding for the protease, and
a sequence coding for inverse transcriptase at least up to codon 340.

17) The method according to claim 15, wherein the amplified product has a size less than about 2800 pb.

18) The method according to claim 15, wherein the amplification of (d1) uses a pair of primers including a sequence of nucleic acids, complementary at 5′ to a phylogenetically conserved region of a gag gene including the cleavage sites, containing all of nucleic acid sequence coding for a protease, complementary at 3′ to a phylogenetically conserved region of a gene coding for the reverse transcriptase.

19) The method according to claim 15, wherein the pair of primers of (d1) includes a sequence of nucleic acids:
complementary at 5′ to a phylogenetically conserved region of a gag gene included between codon 102 of protein p17 (position 1093 on the genome) and codon 76 of protein p24 (position 1415 on the genome), and
complementary at 3′ to a phylogenetically conserved region of a gene coding for inverse transcriptase, included between codon 325 (position 3520) and codon 421 (position 3811 on the genome).

20) The method according to claim 15, wherein the pair of primers of (d1) includes a sequence of nucleic acids:
complementary at 5′ to a phylogenetically conserved region of a gag gene included between codon 126 (position 1165 on the genome) and protein p 17 and codon 21 of protein p24 (position 1250 on the genome), and
complementary at 3′ to a phylogenetically conserved region of a gene coding for inverse transcriptase included between codon 335 (position 3550 on the genome) and codon 395 (position 3751 on the genome).

21) The method according to claim 15, wherein amplification of (d1) is carried out with a pair of primers selected from the group consisting of:
SEQ ID NO. 7 and SEQ ID NO. 9 as a sense primer,
SEQ ID NO. 8 and SEQ ID NO. 10 as an anti-sense primer, and
fragments or analogues of the sequences.

22) The method according to claim 15, wherein the amplification of (d1) is carried out with pair of primers selected from the group consisting of:
a pair of primers with sequences SEQ ID NO. 7 and SEQ ID NO. 8, and
a pair of primers with sequences SEQ ID NO. 9 and SEQ ID NO. 10.

23) The method according to claim 1, wherein the functional analysis of (d3) comprises infecting the HIV target cells with recombinant viruses produced in (d2) in the presence or in the absence of one or several active agents.

24) The method according to claim 1, wherein measuring the replicating capacity of (d4) comprises measuring expression of an indicator gene in response to infection by the recombinant virus of (d2) in comparison to a reference virus.

25) The method according to claim 1, further comprising:
e) processing the data relating to:
possible presence of mutations determined in (c),
functional analysis of the viral proteins of (d2), and
the replicating capacity of (d3), to obtain characteristics of the virus and/or of the treatment when (c) and (d3) and (d4) have been carried out.

26) A set of primers for the method according to claim 1, comprising a pair of primers encompassing a nucleic sequence complementary at 5′ to a phylogenetically conserved region of a gag gene including cleavage sites, containing all of nucleic acid sequence coding for protease and complementary at 3′ to a phylogenetically conserved region of gene coding for inverse transcriptase.

27) The set of primers according to claim 26, wherein the pair of primers encompasses a nucleic acid sequence:
complementary at 5′ to the phylogenetically conserved region of the gag gene included between the codon 102 of protein p17 (position 1093 on the genome) and codon 76 of protein p24 (position 1415 on the genome), and
complementary at 3′ to the phylogenetically conserved region of the gene coding for inverse transcriptase, included between codon 325 (position 3520) and codon 421 (position 3811 on the genome).

28) The set of primers according to claim 26, wherein the pair of primers encompasses a nucleic acid sequence:
complementary at 5′ to the phylogenetically conserved region of the gag gene included between codon 126 (position 1165 on the genome) of protein p17 and codon 21 of protein p24 (position 1250 on the genome), and
complementary at 3′ to the phylogenetically conserved region of the gene coding for inverse transcriptase included between codon 335 (position 3550 on the genome) and codon 395 (position 3751 on the genome).

29) The set of primers according to claim 26, wherein the pair is selected from the group consisting of:
SEQ ID NO. 1, SEQ ID NO. 3 and SEQ ID NO. 5 as a sense primer,
SEQ ID NO. 2, SEQ ID NO. 4 and SEQ ID NO. 6 as an anti-sense primer, and
fragments or analogues of these sequences.

30) The set of primers according to claim 26, wherein the pair is selected from the group consisting of:
a pair of primers R1 of SEQ ID NO. 1 and SEQ ID NO. 2,
a pair of primers R2 of SEQ ID NO. 3 and SEQ ID NO. 4, and
a pair of primers R3 of SEQ ID NO. 5 and SEQ ID NO. 6.

31) A set of primers for the method according to claim 1, comprising a pair of primers selected from the group consisting of:
SEQ ID NO. 7 and SEQ ID NO. 9 as a sense primer,
SEQ ID NO. 8 and SEQ ID NO. 10 as an anti-sense primer, and
fragments or analogues of the sequences.

32) The set of primers according to claim 31, wherein the pair of primers is selected from the group consisting of:
a pair of primers of SEQ ID NO. 7 and SEQ ID NO. 8, and
a pair of primers of SEQ ID NO. 9 and SEQ ID NO. 10.

33) The set of primers according to claim 26, wherein the pair of primers makes it possible to amplify a sequence having a size less than about 2800 pb.

34) A kit comprising at least one set of primers according to claim 26.


SEQUENCE LISTING (10 sequences)

SEQ ID NO: 1 - DNA, 24 bp, Artificial Sequence
  Description of Artificial Sequence Synthetic primer
cctccaggggcaaatggtacatca

SEQ ID NO: 2 - DNA, 30 bp, Artificial Sequence
  Description of Artificial Sequence Synthetic primer
cttgataaatttgatatgtccattggcctt

SEQ ID NO: 3 - DNA, 20 bp, Artificial Sequence
  Description of Artificial Sequence Synthetic primer
tcacctagaactttaaatgc

SEQ ID NO: 4 - DNA, 24 bp, Artificial Sequence
  Description of Artificial Sequence Synthetic primer
ttaaatggctcttgataaatttga

SEQ ID NO: 5 - DNA, 20 bp, Artificial Sequence
  Description of Artificial Sequence Synthetic primer
agccaggtcagccaaaatta

SEQ ID NO: 6 - DNA, 20 bp, Artificial Sequence
  Description of Artificial Sequence Synthetic primer
catgcttcccatgtttcctt

SEQ ID NO: 7 - DNA, 30 bp, Artificial Sequence
  Description of Artificial Sequence Synthetic primer
tggtacatcaggccatatcacctagaactt

SEQ ID NO: 8 - DNA, 28 bp, Artificial Sequence
  Description of Artificial Sequence Synthetic primer
taaatttgatatgtccattggccttgcc

SEQ ID NO: 9 - DNA, 20 bp, Artificial Sequence
  Description of Artificial Sequence Synthetic primer
agaactttaaatgcatgggt

SEQ ID NO: 10 - DNA, 25 bp, Artificial Sequence
  Description of Artificial Sequence Synthetic primer
taaatttgatatgtccattggcctt